(19) United States
(12) Patent Application Publication
(10) Pub. No.: US 2010/0248284 A1
(43) Pub. Date: Sep. 30, 2010

(54) BIOSENSOR

(75) Inventors: Xian Tong Chen, Choa Chu (SG); Min Bin Yu, Choa Chu (SG); Han Hua Feng, Jurong (SG); Quo Qiang Lo, Dover Park View (SG)

Correspondence Address: CONNOLLY BOVE LODGE & HUTZ LLP, 1875 EYE STREET, N.W., SUITE 1100, WASHINGTON, DC, 20006, US

(73) Assignee: AGENCY FOR SCIENCE, TECHNOLOGY AND RESEARCH

(21) Appl. No.: 12/161,681

(22) PCT Filed: Jan. 20, 2006
(86) PCT No.: PCT/SG2006/000012

§ 371 Date: Jun. 15, 2010

(51) Int. Cl. C12Q 1/02; G01N 27/00; H05K 13/00

(52) U.S. Cl. 435/29; 422/82.01; 436/149; 29/825

(57) ABSTRACT

A biosensor which comprises a substrate (101) having a buried electronic sensing element and a substrate surface (124) above the buried electronic sensing element; a structured top layer (125) covering the substrate surface (124), having a top surface above the substrate surface (124), and comprising at least one stimulation and/or sensing electrode (116) and a channel (121) for holding the biomolecule by means of suction through said channel (121) arranged between the top surface (125) and the substrate surface (124), the sensing electrode (116) being electrically coupled to the electronic sensing element; wherein the top surface (125) is provided for placing a biomolecule present in a sample solution thereupon, the sensing electrode (116) is provided for sensing electrical variations in and presence of the biomolecule.

The present invention relates generally to bio-molecular electronics, and more particularly to a biosensor that is used for the detection of a biomolecule, such as a living cell.

### BACKGROUND OF THE INVENTION

Over the past few decades, due to the increasing need for efficient and reliable pre-clinical pharmaceutical drug discovery methods capable of providing a battery of regulatory genotoxicity tests, growing efforts have been channeled towards the development of biosensors that are capable of providing parallel and automated monitoring of ion channel activity in cells. Amongst the various types of biosensors that are currently in use, non-invasive biosensors for carrying out cellular and tissue-based tests have seen rapid development in recent years.

Non-invasive biosensors operate on the principle that the action potential of a cell (or a cellular network) corresponds to the ion currents flowing through ion channels in the cell membrane. It is widely accepted that ion currents are gated or controlled by receptor and channel proteins located within the cell membrane. These proteins are in turn controlled by molecules which can bind to the receptor protein and consequently trigger the opening of ion channels in the cell membrane to enable the flow of ions such as sodium and chloride ions. These ion currents, though small in magnitude, can be accurately measured by field effect transistor (FET)-based, non-invasive biosensors.

In order to study the behaviour of these ion channels, particularly its gating characteristics, a variety of methods can be used to stimulate or modulate the characteristics of the receptor and channel proteins while concurrently monitoring ion currents produced by the cell with a cellular biosensor. These methods include, for example, transmembrane voltage modulation, ligands acting on the intracellular and/or extracellular side of the channel (ligand-gated ion channels), mechanical changes (mechanically-gated ion channels) or combinations thereof.

Some examples of non-invasive FET-based biosensors are described for example in U.S. Pat. Nos. 6,570,196 B1 and 6,602,399 B1, which describe devices that enable direct coupling of ionic signals from a neuron and electronic signals in the semiconductor sensor. This device and method opens up a wide variety of areas of use ranging from basic neurobiological research to high-throughput-screening applications in the pharmaceutical industry. In another example, F. Peter et al. (Physical Review Letters Vol. 76, 327, 1996) describes an experiment in which an AC voltage is applied to a neuron cell by patch clamp device and the resulting signals were recorded by FET-based sensor.

However, the electronic supervision of neuronal nets in these devices requires a precise placement of the neurons on the sensor. A problem encountered in implementing such an arrangement is the establishment of good physical interaction (and thus a stable electrical connection) between a sample and the electrode assembly. One factor contributing to the difficulty in achieving good sample-electrode interaction is the motility and structural plasticity of such cells. Under in vivo conditions, neuronal cells, for example, change their morphology and connectivity during development and in relation to various forms of learning and memory processes. This results in constant changes in the electrical interaction between the cell and the electrode assembly. Such a problem becomes more acute when scaling up the device to provide large scale, high testing throughputs.

Likewise, cultured neurons seldom maintain a constant position on the surface of a test device. Cell displacement has been described, for example, by M. Jenkner, B. Muller and P. Fromherz in Biol. Cybern., Vol. 84, p. 239, 2001 in which the displacement of neuronal cell bodies by neuronal outgrowth is discussed. This neuron mobility results in continuous translocation of the neuronal cell body with respect to the electrode surface which consequently affects the sensitivity of the sensor and the accuracy of the recorded potential. In addition to this problem, a large fraction of cultured neurons often does not form secure physical contact with the sensing device and, thus, a functional contact with the surface electrodes is not established.

Several attempts have been made to address these problems. The PCT patent publication WO 2004/109282 A1 takes advantage of a biological phenomenon known as phagocytosis to anchor a living cell to a structure of protruding metallic micro-nails which function as electrodes. Phagocytosis is an action-dependent process in which the plasma membrane of a living cell extends around a particle, thereby enveloping the particle. The “sinking” of the particle into the living cell leads to the internalization of the particle into the living cell. However, in order for this anchoring to be achieved, the living cell is required to envelope the head of at least one micro-nail to form a tight physical contact. It has been previously reported that such a phenomenon only has a certain statistical chance of occurring, and is consequently not sufficiently reliable for cell monitoring purposes.

G. Zeck and P. Fromherz (Proc. Nat. Acad. Sci. USA, Vol. 98, pp. 10,457 to 10,462, 2001) attempted to overcome the problem of cell displacement by providing a mechanical “fence” in the form of polymeric pillars erected around neuronal cells in order to prevent them from moving away from its initial location. In an experimental setting and for a small number of large neurons, it was reported that such an arrangement was capable of reducing cell displacement. However, in actual practice and in a multi-transistor array, it becomes impractical to manually place individual neurons within micro-scale pillars. For large-scale applications where the speed of operation is essential, it is important for the electronic device to be designed so as to position the neuron automatically and reduce neuronal translocation.

Eversmann et al. (IEEE Journal of Solid-State Circuits, Vol. 38 No. 12 December 2003) discloses a sensor array which uses a CMOS chip for imaging neural activity. The sensing electrode is coated with a biocompatible dielectric layer suitable, and connected to the polysilicon gate of a MOSFET fabricated in a standard CMOS process. The dielectric layer comprises a composite of alternating layers of TiO₂ and ZrO₂. Cell samples to be tested are cultivated directly on the dielectric layer. However, no dedicated structure is provided on the sensor for immobilising the cell samples.

A drawback of the above devices is the difficulty in carrying out pharmaceutical screening of drugs on individual living cells without affecting other adjacent cells. To do so, a pipette patch clamp is needed to bring a test drug substance into contact with the living cell containing a receptor system in order to examine the drug's function as an effector on the cellular receptor system. Such an arrangement requires significant installation effort and a highly qualified operator to manually operate the experimental equipment and is subject to high failure rates. As an example, the device disclosed in F. Peter F. Peter (supra) faces challenges in positioning & stabilizing cells as well as applying AC voltages on cell by patch clamp device.

Therefore, an objective of the present invention is to provide an alternative biosensor that advantageously avoids or reduces some of the abovementioned drawbacks of prior art devices in an economical manner.

### SUMMARY OF THE INVENTION

According to a first aspect of the present invention, a biosensor is provided which comprises a substrate having a buried electronic sensing element for sensing electrical variations due to a change in the status of a biomolecule. The substrate has a substrate surface located above the buried electronic sensing element. A structured top layer is arranged to cover the substrate surface. The structured top layer has a top surface which provides a sensing region on which a biomolecule present in a sample solution is placed. A sensing electrode that is electrically coupled to the electronic sensing element is arranged to have at least a part of its surface exposed to the sensing region. At least one channel is arranged in the structured top layer that is adapted to immobilise the biomolecule by a suction. force, thereby securing the physical contact between .the biomolecule and the sensing electrode.

According to a second aspect of the present invention, a method of forming a biosensor comprises the following steps: providing a substrate having buried therein an electronic sensing element for sensing electrical variations due to a change in the status of a biomolecule, and a substrate surface located above the buried electronic sensing element; covering the substrate surface with a structured top layer, the structured tap layer having a top surface located above the substrate surface; forming in or on the structured top layer a sensing electrode; electrically coupling the sensing electrode to the electronic sensing element; adapting the top surface for placing a biomolecule present in a sample solution thereupon; and forming within the structured top layer at least one channel that is adapted to immobilize a biomolecule by a suction force.

According to a third aspect of the present invention, a method of analyzing the status of a biomolecule is provided, comprising: contacting the sensing electrode of a biosensor of the invention with the biomolecule, measuring a first electrical signal associated with a first status of the biomolecule, exposing the biomolecule to a condition that is suspected to be capable of changing the status of the biomolecule, and measuring a second electrical signal that is associated with the status of the biomolecule after exposure to said condition.

The biosensor of the present invention integrates within a single substrate a FET-based sensing device and the necessary structures that carry out the functions of conventional planar patch clamps using CMOS compatible processes. This integration enables simultaneous and sequential measurements by both conventional patch clamp electrode and FET transistor to be made on a biomolecule, thereby shortening the time required for carrying out sensing applications such as the screening of pharmaceutical substances as well as measurements on the functional characteristics of cell samples. The use of CMOS compatible process for the fabrication additionally facilitates miniaturisation and easy inclusion of with multiple auxiliary structures providing other useful functions. One advantage of the biosensor according to the present invention is the ease of immobilising biomolecular test samples onto the sensing electrode. The suction channel in the invention allows automatic positioning and stabilization of a biomolecule at the sensing electrode and patch clamp electrode, so that a predictable interactions between the biomolecule and the extra-cellular planar potential-sensitive electrode and/or patch-clamp electrode can be achieved. Potential-sensitive electrodes, and hence patch clamp electrodes, can be used simultaneously or sequentially for measurement, thereby eliminating the need for complex procedures described in the prior art for immobilising, stimulating and characterizing the biomolecule.

In this context, the term “biomolecule” refers to biological material including tissue fragments comprising a mass of cells, multi-cell organisms and structures, a single cell and subcellular structures. The term is also used interchangeably with other equivalent terms, such as “bio-molecular body”. Cells to which the invention can be applied generally include any type of cell which is voltage sensitive or cells that are able to undergo a change in its electrical potential, including both eukaryotic cells and prokaryotic cells. Examples of eukayotic cells include both plant and animal cells. Examples of some animal cells include cells in the nervous system such as astrocytes, oligodendrocytes, Schwann cells; autonomic neuron cells such as cholinergic neural cell, adrenergic neural cell, and peptidergic neural cell; sensory transducer cells such as olfactory cells, auditory cells, photoreceptors; hormone secreting cells such as somatotropes, lactotropes, thyrotropes, gonadotropes and corticotropes from the anterior pituitary glands, thyroid gland cells and adrenal gland cells; endocrine secretory epithelial cells such as mammary gland cells, lacrimal gland cells, ceruminous gland cells, eccrine sweat glands cells, and sebaceous gland cells; and other cells including osteoblasts, fibroblasts, blastomeres, hepatocytes, neuronal cells, oocytes, blood cells such as erythrocytes, lymphocytes or monocytes, muscle cells such as myocytes, embryonic stem cells. Other examples of eukaryotic cells include yeast cells and protozoa. Examples of plant cells include meristematic cells, parenchyma cells, collenchyma cells and sclerenchyma cells. Prokaryotic cells applicable in the invention include, for example, archaea cells and bacteria cells. The term biomolecule additionally encompasses other types of biological material such as subcellular (intracellular) structures such as the nucleus, nucleolus, endoplasmic reticulum, centrosome, cytoskeleton, Golgi apparatus, mitochondrion, lysosome, peroxisome, vacuole, cell membrane, cytosol, cell wall, chloroplast, and fragments, derivatives, and mixtures thereof.

The following comments to the design of the biosensor are valid in corresponding form for the method of forming a biosensor.

According to the present invention, a channel is provided for immobilizing the biomolecule to be tested. This channel is formed within a structured top layer, which may comprise any suitable material that can be worked using conventional fabrication techniques. Preferably, the structured top layer comprises a material susceptible to conventional CMOS processing and which thus allows a variety of structures to be formed therein. This structured top layer is formed on top of the substrate surface and has a top surface which provides a sensing region in which a sample solution is placed.

In a preferred embodiment, a channel which terminates in at least two openings located at the top surface is provided, the channel being arranged within the structured top layer, or preferably substantially embedded in the structured top layer, and a first opening of the two openings being located near or proximate to a sensing electrode, and the second of these openings being separated from the first opening. The sensing electrode is arranged to have at least a part of its surface exposed to the sensing region, i.e. in some embodiments, the sensing electrode may be arranged between the top surface of the structured top layer and the substrate surface, or it may be arranged on the top surface of the structured top layer. When the electrode is arranged on the top surface of the structured top layer, measurements on the electrochemical characteristics of the solution or liquid that is in contact with the electrode can be measured, thereby enabling the present invention to be utilised as an electrochemical sensor, for instance. The biomolecule can be immobilised onto the first opening by means of suction in the channel, said suction being exerted through the first opening, thereby generating a low pressure in the channel which results in the movement of sample solution into the first opening and then the channel. This suction force causes the sample biomolecule being tested to move towards the first opening, eventually plugging the first opening and thereby being immobilising over the opening. The sample solution in which the biomolecule is placed may comprise saline, distilled water, or in some instances, nutrient solutions required to keep living cells alive.

The electronic sensing element for sensing electrical variations in the sample is buried within the substrate. The substrate may comprise a single layer or several layers, depending on the desired circuitry for wiring up the electronic sensing element. It would also depend on whether auxiliary electrical components are to be included into the substrate. In one embodiment, the substrate comprises a successively formed layer sequence comprising a semiconductor layer, a first electrically insulating layer, a second electrically insulating layer and a third electrically insulating layer. The sensing electrode connection may be surrounded by an electrically insulated screening electrode, wherein the screening electrode is connected to ground, and wherein the screening electrode is formed in the third electrically insulating layer. The electronic sensing element may also be formed to overlap the semiconductor layer and the first electrically insulating layer.

The electronic sensing element can comprise any electronic device capable of sensing minute changes in the electrical characteristics of the region near to or on the sensing electrode. According to one embodiment, the electronic sensing element is a field effect transistor (FET) having a gate region. In order to establish electrical connection between the biomolecule and the field effect transistor, the sensing electrode is electrically connected to the gate region of the field effect transistor via any suitable connection means. Depending on the embedded position of the field effect transistor within the substrate, connecting structures may be used to physically connect the sensing electrode to the gate region, thereby establish electrical connection between the sensing electrode and the field effect transistor. For example, the sensing electrode may be electrically connected to the gate region of the field effect transistor via a sensing electrode connection.

While measurements can be carried out primarily using the sensing element, other auxiliary components for carrying out sensing functions can be incorporated into the device. In one embodiment, a patch clamp electrode is provided in the channel located next to the first opening and an externally accessible reference electrode provided on the top surface of the structured top layer or any other location in the sensing chamber. These two electrodes can be either connected to external measuring instruments or integrated with the circuits contained in semiconductor layer to measure electrical currents across a cell membrane that is positioned over the first aperture leading to the channel. By positioning a sample biomolecule, e.g. a cell, over the first aperture and applying a suitable suction force on the surface of the cell via the channel, the channel can be made to function as a conventional patch clamp micropipette. Suction force applied can be sufficiently large so that the cell membrane is punctured. Thereafter, measurements on the cell can be carried out using either the sensing element or the patch clamp electrode/reference electrode, or both can be used concurrently to obtain measurements simultaneously.

Apart from providing conventional patch clamp functions, the patch clamp electrode and reference electrode also serves several other functions. Firstly, it detects if a cell positions on or seals the aperture properly. Before a cell position on the aperture, the resistance between a patch clamp electrode and reference electrode is low because electrolyte solution is conductive. When a cell positions itself over or seals the aperture, the resistance will increase. If sealed properly, the resistance should be as high as in the range of around 1 giga-ohms or more, so that the patch clamp test can be successfully reliable performed. A second function of the electrodes is to generate non-uniform electrical field around apertures to assist cells to flow to apertures because of electrophoretic effect. Thirdly, the electrodes characterize electrical properties of trans-membrane ion channels or of ionotropic receptors (for example by voltage clamp techniques). For example, after the membrane of a cell is ruptured, an electrical potential difference is applied across the membrane which contains the respective ion channel or ion channels by patch clamp electrode and reference electrode and simultaneous the current necessary for maintaining this difference is analyzed. In this way, the change in electrical properties of transmembrane ion channel in response to various test stimuli can be measured.

The sensing electrode may have any suitable shape, such as a regular solid elliptical shape or a solid rectangular shape, in particular a solid circular shape or a solid square shape. Alternatively, the sensing electrode may have a regular ring (annular) shape wherein the first opening through which suction force is exerted is located within the ring-shaped sensing electrode. An electrode having such a shape is also known as ring electrodes. It is also possible to use irregular shapes, as long as it provides sufficient contact with the biomolecule to be tested. Apart from sensing electrodes which are unitary-shaped, i.e. comprising a single element, a further alternative which has been contemplated includes a sensing electrode which comprises several individual sensing electrode elements arranged around the first opening of a channel. The sensing electrode elements may have any suitable shape. To improve the likelihood of contact between the biomolecule and the sensing electrode in this alternative, 4 or more sensing electrode elements may be arranged around the first opening of the channel.

In a further embodiment, in addition to the sensing electrode, a stimulation electrode close to opening of the channel is provided to electrically stimulate the sample biomolecule. In the absence of the stimulation electrode, the patch electrodes, if present, may also be employed to exert an electrical stimulus on the sample biomolecule. In either case, the response of individual biomolecules to electrical stimulation can be detected by the sensing electrode and recorded simultaneously.

The electronic sensing element may be electrically connected to external circuits or circuits which are built into the substrate. For establishing electrical connection, the electronic sensing element may be connected to track conductors which are formed in the second electrically insulating layer according to one embodiment. The sensing electrode may be electrically coupled to the electronic sensing element via a sensing electrode connection arranged in the third electrically insulating layer in another embodiment. In between the sensing electrode and the substrate, there may be included auxiliary electrical components such as amplifying circuits for enlarging the signal obtained by the electronic sensing element.

In one embodiment, the auxiliary electrical component comprises screening electrodes which are connected to ground. The purpose of the grounded screen electrode is to screen any noise signals coming from sensing circuits, stimulation circuits and amplifying circuits in the substrate, particularly in the semiconductor layer during positioning, or during patch clamp test or when stimulating cells. If the electronic sensing element is a field effect transistor (FET), then the source and drain regions of the FET are both arranged in the semiconductor layer and connected to the track conductors. Further, the gate region of the FET is electrically connected to the sensing electrode via the sensing electrode connection. For preventing undesired short-circuits, most of the layers of the substrate are made of electrically insulating material except of the semiconductor layer which is used for forming the source and drain regions as well as necessary circuits such as amplifying, .recording circuits. The gate region is preferably electrically insulated from the channel region by a part of the first electrically insulating layer.

In another embodiment, the top surface of the structured top layer, including the surface of the sensing electrode which forms part of the top surface of the structured layer, is covered with a bio-compatible layer. In instances where improved attachment of the biomolecule onto the sensing electrode and/or top surface of the structured top layer is required, this bio-compatible layer may be provided. Additionally, such a bio-compatible layer prevents the biomolecule from absorbing contaminants such as toxic substances from any layer which comes into contact with it. Examples of a bio-compatible layer which can be used includes Collagen (Types I, III, or V), Chitosan, Heparin, as well as additional components such as Fibronectin, Decorin, Hyaluronic Acid, Chondroitin Sulphate, Heparan Sulphate and growth factors (such as TGFβ, bFGF).

Since bio-compatible materials are typically electrically insulating, this bio-compatible layer is preferably sufficiently thin to ensure that the sensitivity of the sensing electrode is not affected, i.e. it is still able to detect any electrical potential that is induced by the biomolecule. The sensing electrode either may be in direct electrical conducting contact with the biomolecule to be sensed and analyzed, or may be capacitively coupled to the biomolecule to be sensed and analyzed via a bio-compatible layer. If desired, an additional electrical insulating layer may be provided between the top surface and the biomolecule.

In one embodiment, the top surface of the biosensor is provided with chamber walls. The chamber walls may be arranged to define at least one sensing chamber, or for large scale applications, several sensing chambers in which a sample to be tested is placed. In one embodiment, the first opening of the channel is formed inside the at least one sensing chamber, and a second opening of the two openings of the channel is formed outside the at least one sensing chamber.

In a presently preferred embodiment, the chamber walls are arranged to define, in addition to the sensing chamber, at least one fluid control chamber within which the second opening of the channel is formed. In this embodiment, the fluid control chamber serves to hold fluid that has been drained out or discharged from the sensing chamber. For example, if the biomolecule is comprised in a sample solution and is placed within the sensing chamber, sample solution can be drawn from the sensing chamber into the fluid control chamber through the channel through suction. This results in the biomolecule being drawn towards the opening and eventually positioned over the opening until it ‘plugs’. the opening, thereby becoming immobilised at the openings as explained above.

According to the second aspect of the invention, a method is provided for forming a biosensor of the invention. The method comprises providing a substrate having a buried electronic sensing element, covering the substrate surface with a structure top layer, forming in or on the structured top layer a sensing electrode and a channel for immobilizing a sample biomolecule, electrically coupling the sensing electrode to the electronic sensing element and adapting the top surface for placing thereupon a biomolecule. An advantage provided by the method of the invention is that it enables a lateral channel to be formed within the structured top layer without the use of machining equipment.

In one embodiment, the method of the invention uses a systematic combination of conventional CMOS fabrication techniques, including wet etching, chemical-mechanical polishing, and material deposition as described in greater detail below. The channel may be formed in the structured layer, having at least two openings terminating at the top surface, the channel being substantially buried in the structured top layer, a first opening of the two openings (preferably having a diameter ranging from about 0.3-5 μm to about 10 μm, which is within a range that should be substantially smaller than the biomolecule to be tested) being arranged next to the sensing electrode. The function of the channel is to provide hydraulic control of the position of the biomolecule placed above the sensing electrode through suction. Draining of the sample solution through the channel generates a suction force at the first opening. This suction force is used to hold the biomolecule in place above the sensing electrode.

The channel can be formed by conventional CMOS backend processes, such as film deposition, photo-lithography, dry etching, CMP and wet etch. It is crucial to choose optimal combination of sacrificial material for channel and surrounding material such that sacrificial material can be removed by wet etch process while surrounding material is kept intact during and after wet etch process.

The third aspect of the invention is relates to specific uses of the biosensor of the invention. In general, the biosensor of the invention may be used in any application involving the electrophysiological evaluation of biomolecules. Such applications typically require contact between the biomolecule being evaluated and a electronic sensor, such as a transistor. Common applications for the biosensor include the screening of drugs (e.g. electrophysiological determination of compound activity on ion channels in cell membranes is studied) and studies into the characteristics of cells (studies on the mechanisms of microelectrode electroporation).

In cell studies, e.g. in studies to characterise membrane polarisation or to determine trans-membrane threshold potential for pore formation or detection of the occurrence of a change in the status of the biomolecule can be made by comparing the first and/or the second electrical signals to pre-determined threshold electrical signal values. For example, the second electrical signal may be compared against a known electrical signal that is known to correspond to a changed status; alternatively, the shape and magnitude of the difference between the first and the second electrical signal may be compared to the pre-determined threshold electrical signal value. When the shape and magnitude of the difference between the first and the second electrical signal is larger than those of the pre-determined threshold electrical signal value, the condition to which the biomolecule is exposed is determined to be capable of changing its status.

In one embodiment, the cell to be tested is positioned on the predetermined test site by suction force exerted through the channel, and then the surface of the biomolecule is ruptured by means of the suction force (which may have the same or different magnitudes as the suction force used to immobilize the biomolecule), thereby allowing the transmembrane electrical properties of ion channels or of ionotropic receptors to be accessed. In order to carry out electrical measurements on the sample, an AC electrical potential difference is applied by the patch clamp electrode and the reference electrode across the biomolecule, the surface of which contains ion channels, and simultaneously the resulting electrical current (derived from ion movement through a transport structure located within or isolated from the region of the cell on which the suction force is applied) necessary for maintaining this difference is measured by FET transistor. In this respect, transport structures in a cell include any of the following structures located in a cell membrane: anion channels, cation channels, anion transporters, cation transporters, receptor proteins and binding proteins. In accordance with conventional patch clamp techniques, the above measurement may be carried out on an intact cell using the whole cell or cell attached approach.

In the course of carrying out the above mentioned applications, it is preferable to immobilise the biomolecule onto a surface of the sensing electrode in order for it to be in contact with an electronic sensing device. In one embodiment, immobilization of the biomolecule onto the biosensor is performed by means of suction force that is generated at the top surface of the biosensor. Suction force can be generated via pump-driven suction of sample solution through one or more openings in the sensing chamber in which the biomolecule is placed, the apertures being connected to a channel that removes the sample solution from the sensing chamber.

Drugs and medicinal food substances that are suspected to have a therapeutic effect on humans or animals when consumed can be screened using the biosensor of the invention. For this purpose, the following steps may be carried out: measuring a first electrical signal associated with a first status of the biomolecule, exposing the biomolecule to the screening substance that is suspected to be capable of changing the status of the biomolecule, and measuring a second electrical signal that is associated with the status of the biomolecule after exposure to said screening substance. By carrying out the above method, the effect of the screening substance on cells, especially on functional characteristics of ion channels in cells, can be can be measured.

When using the biosensor to carry out conventional patch clamp measurements on a biomolecule, patch clamp electrode and reference electrode that are connected to a patch clamp circuit are used both to control the potential and to measure the currents conducted across the membrane. The current or electrical potential and their change in response to stimulus can be measured by the reference electrode present at the top surface of the biosensor which is in contact with the sample solution and the electrode in the channel. The stimulus that is applied to the biomolecule may comprise rupturing the surface of the biomolecule by means of the suction force and producing electrical continuity between the electrolyte in the channel and interior of the cell, thereby allowing total current that flow through various ion channel or voltage to be measured by the patch clamp electrode located in a channel and reference electrode present at the top surface of the biosensor.

Therefore, the present invention provides an apparatus and methods

1) to simplify positioning of a biomolecule, in particular to position the biomolecule automatically and precisely, 2) to overcome displacement problems of the biomolecule, e.g., of a neuron on a surface of an electronic device such as a field effect transistor (FET), by stabilizing the biomolecule at a predetermined extra-cellular planar potential-sensitive electrode, and 3) to easily screen a test substance for the above mentioned biomolecule. In certain embodiments, the present invention can be employed to screen a plurality of bio-molecular bodies simultaneously.

The present invention can be used for positioning and electrically analyzing of a biomolecule. Positioning a biomolecule, as used herein, generally comprises locating or placing the biomolecule at a predetermined position within the system, typically for subsequent analysis. Analyzing the biomolecule, as used herein, generally comprises detecting the presence of and the activity within the biomolecule, while it is positioned at the predetermined position, typically with respect at least in part to the electrical properties of the biomolecule.

The various aspects of the invention will be more fully understood in view of the following description, drawings and non-limiting examples.

### BRIEF DESCRIPTION OF THE DRAWINGS

In order to understand the present invention and to demonstrate how the present invention may be carried out in practice, preferred embodiments will now be described by way of non-limiting examples only, with reference to the accompanying drawings, in which:

FIG. 1 shows the cross-sectional view of a biosensor according to an exemplary embodiment of the present invention;

FIG. 2A to FIG. 2J show each a cross-section through the biosensor according to the second embodiment of the present invention during different production steps; and

FIG. 3A shows a top view onto a biosensor according to a third embodiment of the present invention in which a ring shaped sensing electrode is present in the biosensor. A cross-sectional view of a section of this embodiment along the dotted line shown in FIG. 3A is illustrated in FIG. 3B. FIG. 3C shows a simplified diagram showing how the suction channel can be connected to a supply channel for delivering a screening substance to the sample biomolecule. FIG. 3D shows a top view of another embodiment of the invention in which horse shoe-shaped sensing electrodes is used.

FIGS. 4, 5, 6 depict a cross sectional view of one embodiment of the biosensor which includes a stimulation electrode for electrocuting a sample biomolecule. FIG. 5 shows the use of only the patch clamp electrode disposed in the channel, while FIG. 4 shows the use of only the stimulation electrode. FIG. 6 shows the use of both electrodes for providing simultaneous or concerted electrocution of the biomolecule.

FIG. 7A to 7C show one embodiment in which an array of sensing electrodes and stimulation electrodes are used to evaluate a set of cells, and a screening substance is applied to a targeted cell in isolation.

FIG. 8A to 8C show another embodiment in which an array of sensing electrodes and stimulation electrodes are used to evaluate a set of cells, and a screening substance is added to the sensing chamber, thereby affecting all the cells within the sensing chamber.

FIGS. 9, 10 and 11 shows various embodiments of the sensing electrode and their corresponding layout in the biosensor.

### DETAILED DESCRIPTION

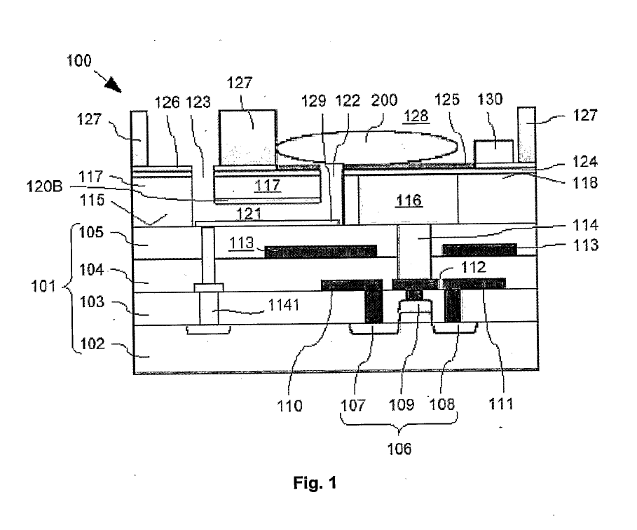

A cross-section through a biosensor 100 according to a first embodiment of the present invention is shown in FIG. 1. A biomolecule 200, according to the first embodiment a living cell such as a neuron, is placed on the biosensor 100 for analysis. According to the first embodiment of the present invention, the biosensor 100 detects the electrical properties of the biomolecule 200 by means of a field effect transistor (FET) 106 which is buried in a substrate 101.

The substrate 101 is a layer sequence comprising, successively, a semiconductor layer 102, a first electrically insulating layer 103, a second electrically insulating layer 104 and a third electrically insulating layer 105. The layer sequence of the substrate 101 terminates with a substrate surface 115 confining the third electrically insulating layer 105. It is preferred to use silicon (Si) as material for the semiconductor layer 102, and to use silicon dioxide (SiO₂) as material for the first, second and third electrically insulating layers 103, 104, 105. Nevertheless, any other suitable semiconductor and electrically insulating materials, respectively, can be used for the layers 101 to 105 in the substrate 101.

The FET 106 comprises source and drain regions 107, 108 arranged in the semiconductor layer 102 and formed by suitable doping of the semiconductor layer 102, and a gate region 109 arranged in the first electrically insulating layer 103 above and laterally between the source and drain regions 107, 108 such that a remainder of the first electrically insulating layer 103 is maintained between the gate region 109 and the semiconductor layer 102 for electrical insulation of the gate region 109. In the semiconductor layer 102, the region between the source and drain regions 107, 108 below the gate region 109 acts as channel region of the FET 106. The source and drain regions 107, 108 are electrically connected with respective source and drain track conductors 110, 111 which are arranged in the second electrically insulating layer 104. The gate region 109 is electrically connected with a gate lead 112 which is also arranged in the second electrically insulating layer 104. Further, the gate region 109 is electrically connected to a sensing electrode 116 via a sensing electrode connection 114 and the gate lead 112. The sensing electrode 116 is arranged on the substrate surface 115 above the sensing electrode connection 114 which itself is arranged above the gate lead 112 and extends from the gate lead 112 through the second and third electrically insulating layers 104, 105 to the sensing electrode 116. It is preferred to use copper (Cu) as material for the sensing electrode 116 and for the sensing electrode connection 114. Nevertheless, any other suitable electrically conducting materials can be used for the sensing electrode 116 and/or for the sensing electrode connection 114. Further, an electrically conducting material can be used for the sensing electrode 116 which is different to the electrically conducting material used for the sensing electrode connection 114.

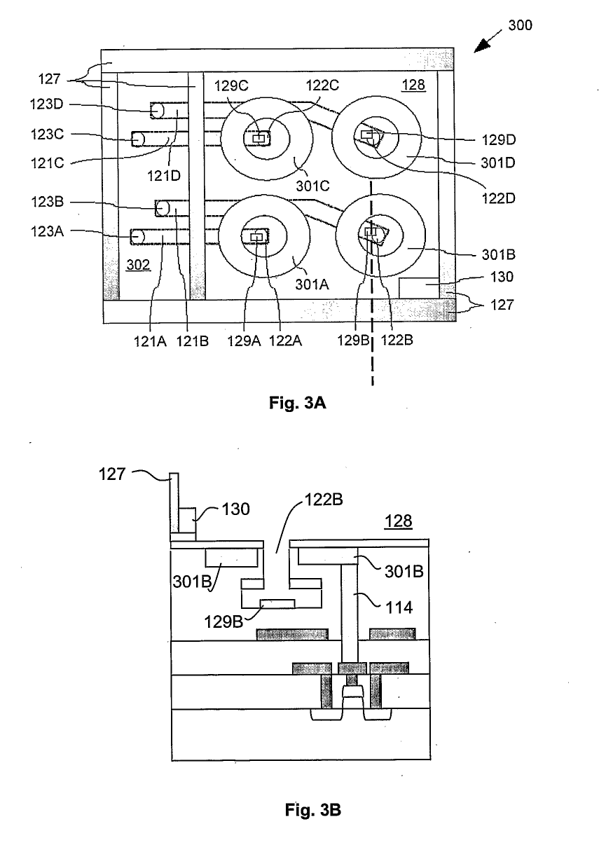

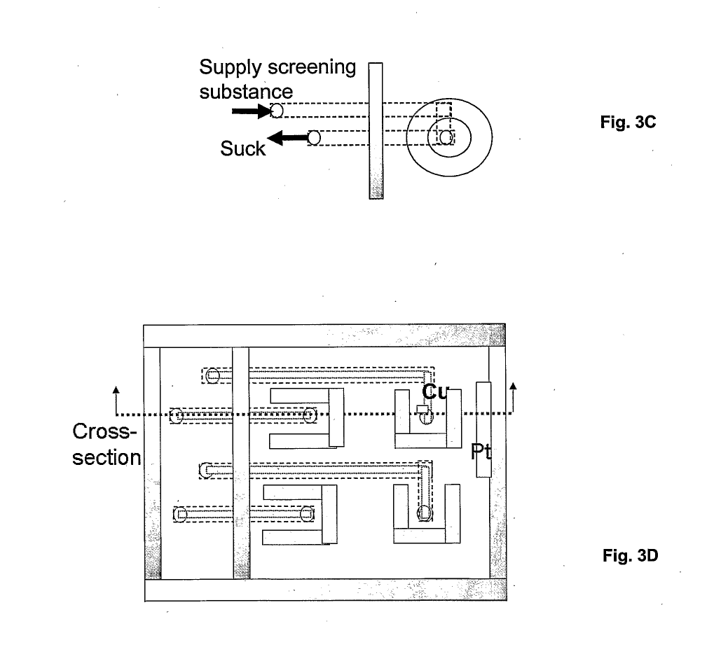

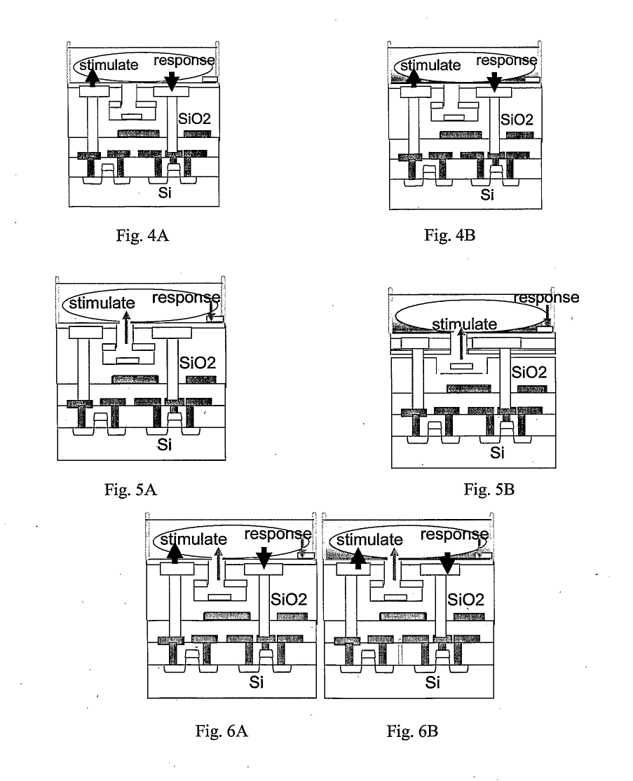

In top view, the sensing electrode 116 may have any suitable shape. According to the first embodiment of the present invention, the shape of the sensing electrode 116 in top view is a solid square. According to a third embodiment of the present invention which is shown in top view in FIG. 3A, the sensing electrodes 301A to 301D have the shape of a ring. Alternative shapes of the sensing electrode 116 of the first embodiment of the present invention in top view are solid rectangular, solid elliptical and solid circular as described earlier.

There is buried an electrically insulated screening electrode 113 around the sensing electrode connection 114 in the third electrically insulating layer 105 adjacent the interface between the second and third electrically insulating layers 104, 105. This screening electrode 113 is electrically connected to ground and protects the FET 106 against undesired capacitive influences impinging through the substrate surface 115. In other embodiments of the present invention, this screening electrode 113 may be omitted without departing from the essence of the present invention. The substrate surface 115 is covered with a structured top layer 117 having a top surface 118 opposite the substrate surface 115, wherein the sensing electrode 116 is contained in the structured top layer 117 and extends from the top surface 118 to the substrate surface 115.

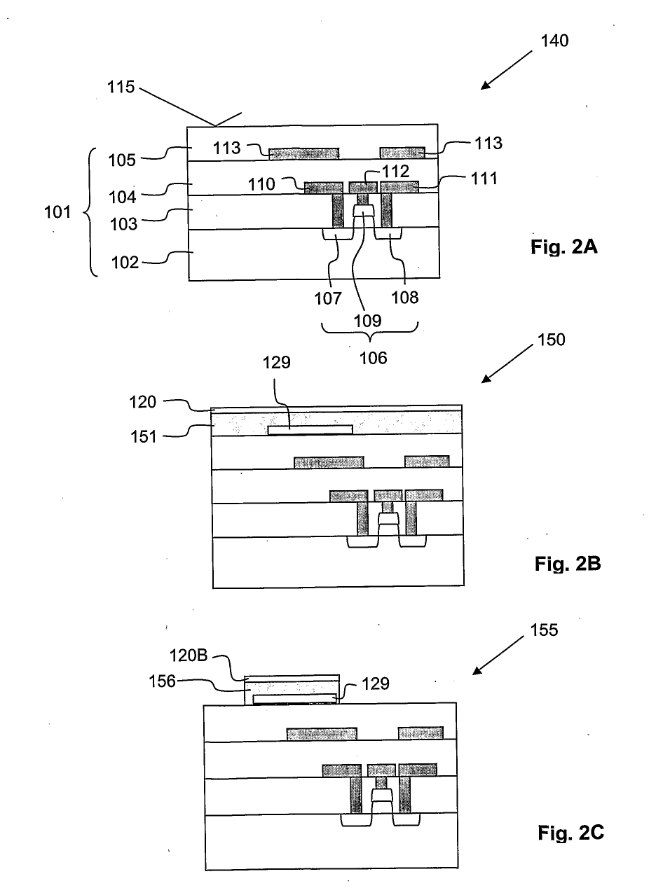

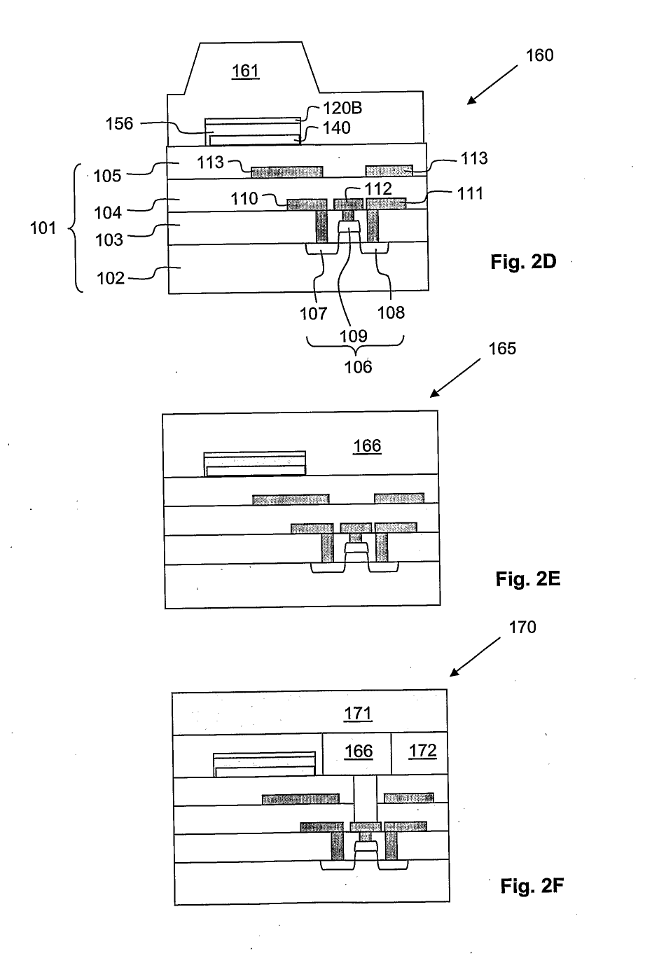

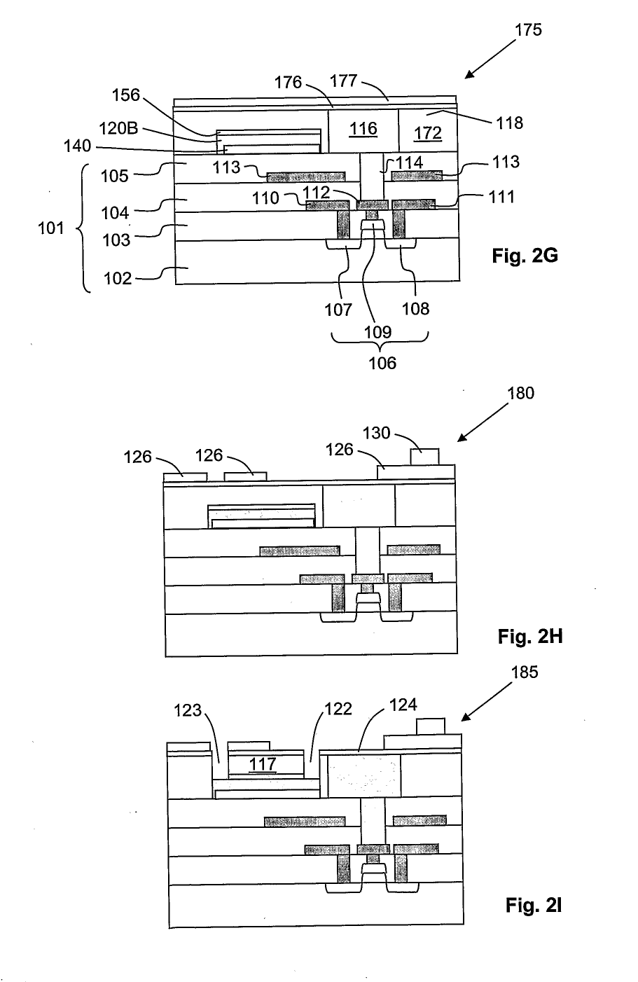

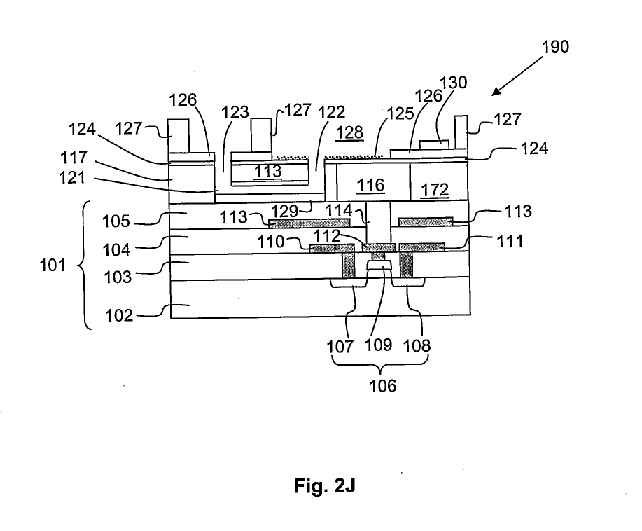

As shown in FIG. 1, a channel 121 extends through the structured top layer 117 from a first opening 122 to a second opening 123, wherein both the first and the second openings 122, 123 terminate at the top surface 118, and wherein the first opening 122 is arranged adjacent the sensing electrode 116. Therefore, the channel 121 is substantially U-shaped having a wide basis and two short legs. The top wall of the basis of the channel 121 is covered with a second part of the first etch mask layer 120B remaining from the production process (described with respect to FIG. 2A to FIG. 2J). This channel 121 provides for positioning and stabilizing the biomolecule 200 by hydraulic control of the flow of the sample solution (which may comprise nutrients for the biomolecule 200, for example). Further, the biomolecule 200 can be attached to the first opening 122 via suction if low pressure suction is generated in the channel 121 and exerted through the first opening 122 to draw the sample solution out of the sensing chamber via the first opening. This enables the biomolecule 200 to be fixedly positioned and stabilized at the opening, while maintaining good contact with the sensing electrode. The suction of the biomolecule 200 to the first opening 122 is comparable with the fixation during conventional patch clamping using a pipette.

The top surface 118 of the structured top layer 117 is covered with a thin, cap dielectric layer 124 which should be bio compatible and possess (1) high dielectric constant to improve capacitive coupling, (2) resistance to cell culture solution and (3) good barrier performance for metal sensing electrode. The first and second openings 122, 123 remain uncovered. The cap dielectric layer 124 is made of silicon nitride (SiN), for example. This cap layer 124 is partly covered with a binding layer 125 for improving binding of the biomolecule 200 to the biosensor 100. Further, the binding layer 125 acts as bio-compatible layer for the biomolecule 200 to prevent toxic substances possibly being present in the biosensor 100 from being absorbed by the biomolecule 200, amongst several other functions. The binding layer 125 is made of, for example, a 0.1% poly-L-Lysine (Sigma). The remainder of the cap layer 124 is covered with a fourth electrically insulating layer 126 made of silicon dioxide (SiO₂), for example, to improve adhesion of chamber walls 127.

A plurality of bio-compatible chamber walls 127 is provided above the fourth electrically insulating layer 126. These bio-compatible chamber walls 127 form at least one sensing chamber 128. Each sensing chamber 128 comprises at least one each of the sensing electrode 116 covered with the binding layer 125 and having arranged the first opening 122 of the channel 121 next to the sensing electrode 116. Each sensing electrode 116 is provided for sensing a single biomolecule 200 present in the sensing chamber 128. Due to the provision of the binding layer 125 and due to the fact that the bio-compatible chamber walls 127 are made of bio-compatible material, a biomolecule 200 can easily be immobilized, i.e., positioned, above the sensing electrode 116 and can be held there by means of the channel 121 having a low pressure therein in a stable position for sensing non-invasively by means of the sensing electrode 116, the sensing electrode connection 114 and the FET 106.

Further, each sensing chamber 128 comprises a patch clamp electrode 129 in the channel 121 next to the first opening 122 and a reference electrode 130 arranged on the fourth electrically insulating layer 126 for automatically conducting patch clamp tests without the need of an operator to place a patch clamping pipette onto the biomolecule 200. The patch clamp electrode 129 may be connected to a voltage source via electrode connector 1141. The electrode connector is in physical contact with both the sensing electrode and the electronic sensing element, thereby electrically coupling the sensing electrode to the electronic sensing element. Accordingly, patch clamp tests conducted by means of the patch clamp electrode 129 and the reference electrode 130 are carried out on an intact cell using the whole cell or cell attached approach. The bio-compatible chamber walls 127 are made of the organic polymer (i.e., the elastomer) polydimethylsiloxane (PDMS) which is bio-compatible. The patch clamp electrode 129 and the reference electrode 130 are preferably made of platinum (Pt).

It is pointed out that the materials used for the biosensor 100 and described above shall not be understood as limiting, other materials which correspond to the materials mentioned above can be used in like manner.

Cross-sections through the biosensor 100 according to the second embodiment of the present invention during different production steps are now shown in FIG. 2A to FIG. 2J. Features already described with respect to FIG. 1 will not be described again here. Nevertheless, same reference signs refer to identical components.

The production of the biosensor 100 is described starting at a first intermediate product. 140 shown in FIG. 2A fabricated with generally known CMOS technology such as implant, film deposition, photolithography, dry etch and CMP. This first intermediate product 140 comprises the substrate 101 with buried FET 106, buried source and drain track conductors 110, 111, buried gate lead 112, and buried screening electrode 113. As already mentioned above, the substrate 101 is bordered above the FET 106 with the substrate surface 115. This first intermediate product 140 is produced using generally known production processes, a description thereof is omitted here.

On the substrate surface 115 of the first intermediate product 140, there is produced the patch clamp electrode 129 made of Pt using generally known lift-off methods. Then, the entire remaining substrate surface 115 together with the patch clamp electrode 129 is covered with a complete vertically thick organic layer 151, which can be made of the semiconductor dielectric resin named SiLK™ which is an organic spin-on polymer with a dielectric constant of 2.6 distributed by The Dow Chemical Company and the first etch mask layer 120. This is done by conventional layer deposition processes. A second intermediate product 150 as shown in FIG. 2B results.

Thereafter, the second intermediate product 150 is patterned and etched by conventional layer patterning and etching processes to remove parts of the first etch mask layer 120 and of the complete vertically thick organic layer 151. At the same time, the second part of the first etch mask layer 120B arranged on a remaining vertically thick organic layer 156 is maintained. This results in a third intermediate product 155 as shown in FIG. 2C.

Now, a thick electrically insulating layer 161 is deposited on the third intermediate product 155. The thick electrically insulating layer 161 is formed by depositing undoped silicate glass (USG) in a uniform deposition process and, therefore, comprises silicon dioxide (SiO₂). Since a uniform deposition process is used, the thick electrically insulating layer 161 has an exposed surface comprising elevations and depressions depending on the number and distribution of layers beyond the thick electrically insulating layer 161. The third intermediate product 155 together with the thick electrically insulating layer 161 forms a fourth intermediate product 160 which is shown in FIG. 2D.

The elevations and depressions being present at the fourth intermediate product 160 are leveled by chemical-mechanical polishing (CMP) resulting in a leveled electrically insulating layer 166 and, therefore, in a fifth intermediate product 165 (compare FIG. 2E).

In the following, a dual-damascene process is applied to the fifth intermediate product 165 comprising pattering and etching the fifth intermediate product 165, i.e. the leveled electrically insulating layer 166 and the second and third electrically insulating layers 104, 105 are etched, and depositing copper (Cu) on and in the etched fifth intermediate product 165 for producing the sensing electrode connection 114 and the sensing electrode 116. Usually, copper is over-deposited in such a dual-damascene process such that a copper layer 171 covering the whole etched and filled fifth intermediate product 165 remains. This etched and filled fifth intermediate product 165 together with the copper layer 171 form a sixth intermediate product 170 which is shown in FIG. 2F. During etching, the leveled electrically insulating layer 166 is changed to an etched electrically insulating layer 172.

Afterwards, the copper layer 171 is removed by chemical-mechanical polishing (CMP) until the etched electrically insulating layer 172 is exposed, thereby forming the top surface 118. Thereafter, the top surface 118 is covered firstly with an unstructured cap layer 176 and secondly with an unstructured fourth electrically insulating layer 177. Therefrom, a seventh intermediate product 175 results (compare FIG. 2G).

Further, the reference electrode 130 is formed on the unstructured fourth electrically insulating layer 177 by a commonly known lift-off process. After that, the unstructured fourth electrically insulating layer 177 is structured by means of etching to form the fourth electrically insulating layer 126 and to expose the unstructured second etch mask layer 176 for later structuring. This yields an eighth intermediate product 180 which is shown in FIG. 2H.

Now, the eighth intermediate product 180 is patterned and suitable etch processes are subsequently applied to the eighth intermediate product 180 to form the first and second openings 122, 123 of the channel 121. Therefore, the unstructured second etch mask layer 176 is structured, i.e., partly removed, such that the second etch mask layer 124 results. Further, the first and second openings 122, 123 structure the etched electrically insulating layer 172 to yield the structured top layer 117. The eighth intermediate product 180 together with the first and second openings 122, 123 and the second etch mask layer 124 forms a ninth intermediate product 185 as shown in FIG. 2I.

For completion of the biosensor 100 (compare FIG. 2J), the ninth intermediate product 185 is wet etched such that the remaining vertically thick organic layer 156 is removed. Therefore, the channel 121 is completed, i.e. the first and second openings 122, 123 are interconnected. During this wet etch process, the patch clamp electrode 129 is not affected. Then, the bio-compatible chamber walls 127 are formed on appropriate portions of the fourth electrically insulating layer 126 from bio-compatible material by generally known formation processes. According to the first embodiment of the present invention, polydimethylsiloxane (PDMS) is used as bio-compatible material for the bio-compatible chamber walls 127. Finally, the exposed parts of the second etch mask layer 124 which are not covered by the fourth electrically insulating layer 126 are covered with the binding layer 125. Please note that for covering the exposed parts of the second etch mask layer 124 with the binding layer 125, a selective deposition process is applied. To this end, the materials of the second etch mask layer 124 and of the fourth electrically insulating layer 126 should be suitably selected.

A plurality of the bio-compatible chamber walls 127 arranged around the binding layer 125 form the sensing chamber 128, wherein the sensing chamber 128 comprises at least one sensing electrode 116 covered with the binding layer 125 and having arranged the first opening 122 of the channel 121 next to the sensing electrode 116. The sensing electrode 116 and the sensing electrode connection 114 are provided for sensing a single biomolecule 200 by means of the FET 106.

A top view onto a biosensor 300 according to a third embodiment of the present invention is shown in FIG. 3A. A cross-sectional view of a section of this embodiment along the dotted line shown in FIG. 3A is illustrated in FIG. 3B. Components which are identical in the first and second embodiments of the present invention are denoted therein with identical reference signs.

The biosensor 300 differs from the biosensor 100 according to the second embodiment of the present invention in three main differences:

A first difference is that the biosensor 300 of the third embodiment comprises four sensing electrodes 301A, 301B, 301C, 301D in the sensing chamber 128, whereas the biosensor 100 of the second embodiment comprises only one sensing electrode 116 in the sensing chamber 128. It is understood that as exemplified in the third embodiment that the present invention is scalable to any suitable size by fabricating a matrix of sensing chambers and corresponding sensing electrodes. Any regular arrangement of any number of sensing electrodes would be possible in other embodiments of the present invention. Further, the number of sensing electrodes comprised in a single sensing chamber and, in like manner, the total number of sensing chambers and the arrangement of the sensing chambers with respect to each other may be varied as desired.

A second difference is that each of the four sensing electrodes 301A, 301B, 301C, 301D comprises a ring-shape in top view onto the biosensor 300 of the second embodiment, whereas the sensing electrode 116 of the biosensor 100 of the first embodiment comprises in top view a solid square shape. The ring-shape of the four sensing electrodes 301A, 301B, 301C, 301D leaves in each case a central area unfilled by material of the corresponding one of the four sensing electrodes 301A, 301B, 301C, 301D.

A third difference is that in the second embodiment, the first openings 122A, 122B, 1220, 122D are arranged each in the central area of the respective one of the four sensing electrodes 301A, 301B, 301C, 301D, whereas in the first embodiment the first opening 122 is arranged beside but near to the sensing electrode 116. Above the exact arrangement of the first opening with respect to the corresponding sensing electrode, it is essential for the present invention that each sensing electrode has in close proximity a first opening for holding the respective biomolecule.

The first openings 122A, 122B, 122C, 122D are connected via corresponding channels 121A, 121B, 121C, 121D to second openings 123A, 123B, 123C, 123D, respectively.

Adjacent to the sensing chamber 128 and separated from the sensing chamber 128 by means of merely one chamber wall 127 there is arranged a fluid control chamber 302 through which fluid from the sensing chamber 128 is discharged. The second openings 123A, 123B, 123C, 123D terminate all together in the fluid control chamber 302 which enables fluid control of the sample solution provided for the nutrition of the bio-molecular bodies 200. This fluid control is further used for generating a low pressure in the channels 121A, 121B, 121C, 121D such that sample solution containing a biomolecule 200/bio-molecular bodies 200 being present above (one of) the four sensing electrodes 301A, 301B, 301C, 301D is/are drawn out, thus resulting in the biomolecule 200 being drawn towards the opening and eventually positioned over the opening to ‘plug’ the opening, thereby establishing a stable hold at the respective (one of the) first openings 122A, 122B, 122C, 122D.

Each channel 121A, 121B, 121C, 121D comprises a patch clamp electrode 129A, 129B, 129C, 129D (each of which can have a portion thereof which is extended to 2nd opening 123A, 123B, 123C, 123D as shown in FIG. 3D), respectively, and patch clamp tests can be carried out for each patch clamp electrode 129A, 129B, 129C, 129D separately with respect to only one reference electrode 130 positioned in the sensing chamber 128. According to the second embodiment of the present invention, the reference electrode 130 is arranged at a corner of two chamber walls 127, but any other suitable arrangement of the reference electrode 130 is also possible.

A further embodiment of the biosensor is shown in FIG. 3D in which the biosensor 400 comprises C-shaped electrodes instead of ring electrodes. This embodiment also comprises a fluid control chamber 302. The inlets 122 to the respective channels 121 are arranged at approximately at the centre in the hollow of the electrode.

FIGS. 4A and 4B describe a simple application using the biosensor of the invention to investigate response of a cell to electrical and chemical stimulation using the transistor as a sensor. In this embodiment, the biosensor is provided with a stimulation electrode to stimulate the cell, thereby allowing observations on the response behaviour of the cell. As can be seen in FIG. 4A, the response of the cell to the electrical stimulus was tested prior to the application of screening substance. After applying screening substance to the cell nutrients, the response of the cell was tested (FIG. 4b). This second reading was compared to the first reading to evaluate the effect of screening substance on ion channels.

FIGS. 5A and 5B illustrate a patch clamp test that is carried out on a cell to evaluate the response of a cell to chemical stimulation using the patch clamp electrodes located in the sensing chamber and in the suction channel. The membrane at the first aperture is ruptured by increasing suction force. Prior to addition of a screening substance to the cell nutrient, an initial reading was taken from electrodes in the suction channel and the sensing chamber. After the addition of screening substance, a second reading was taken and compared to the initial reading to evaluate the response of the cell to chemical stimulation.

FIGS. 6A and 6B show an ac voltage that is applied to the neuron by patch clamp electrode and the resulting signals were recorded by FET transistor before (FIG. 6A) and after (FIG. 6B) addition of screening substance to the cell nutrient respectively. An additional screening substance which may be different from the previous one can also be supplied to the cells through the channel without affecting the other cells to be tested. A combination of readings of the cell response to stimulation from both stimulation electrodes and from the patch clamp electrodes can also be taken, as shown in FIGS. 6A and 6B.

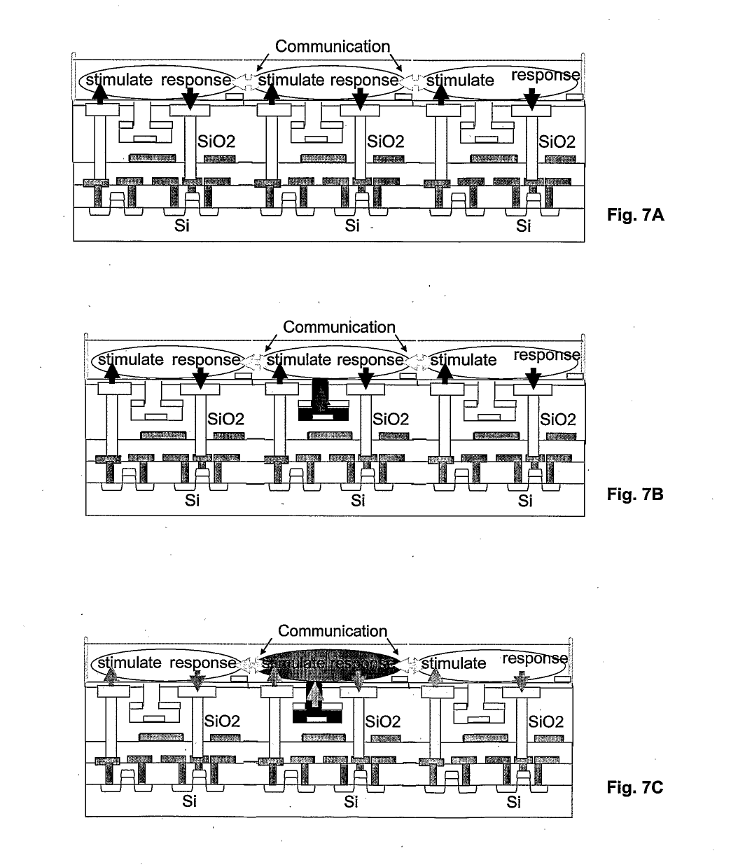

FIGS. 7A to 7C show cross-sectional views of a biosensor array having 3 sensing electrodes arranged adjacent to each other within the sensing chamber. The figure is a diagrammatic scheme of the communication mechanism between cells and the effect of screening substance on a sample biomolecule (a) before, (b) after the addition of the screening substance onto a target cell (centre cell) by measuring electrical variance in response to stimulus, and (c) where screening substance is injected directly into the cell. In this embodiment, the cells to be tested except centre cell will not be affected by the screening substance.

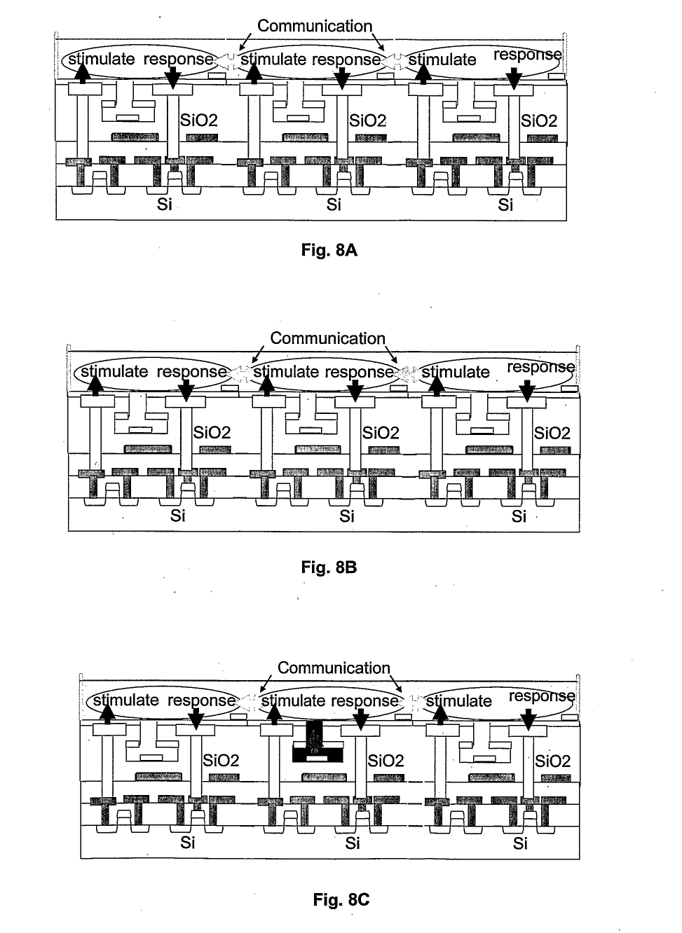

FIGS. 8A to 8C show another set of cross-sectional views of a biosensor having 3 sensing electrodes arranged adjacent to each other within the sensing chamber. The figure is a diagrammatic scheme of the communication mechanism between cells and the effect of screening substance on a sample biomolecule (a) before, (b) after the addition of the screening substance onto into the entire sensing chamber thereby affecting all the cells within the sensing chamber, and (c) where screening substance is injected directly onto or into the cell. For example, a blood sample may contain a known virus, and the screening substance may comprise a drug suspected to bring be effective against the virus.

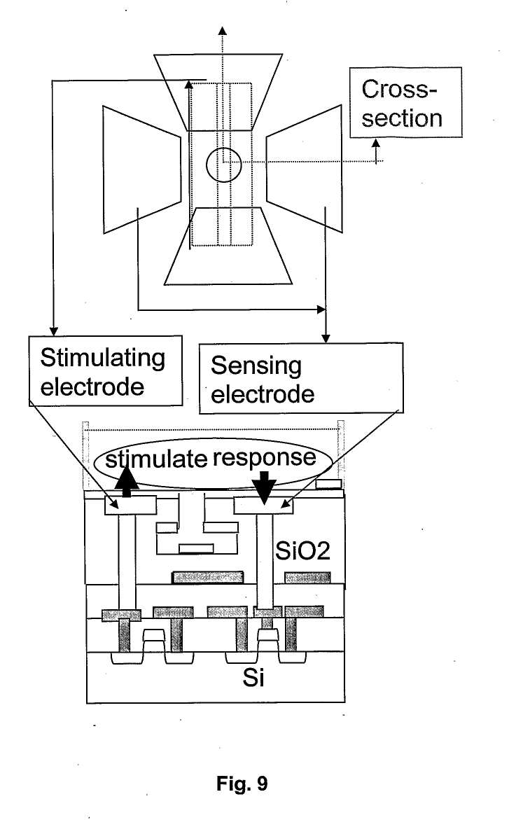

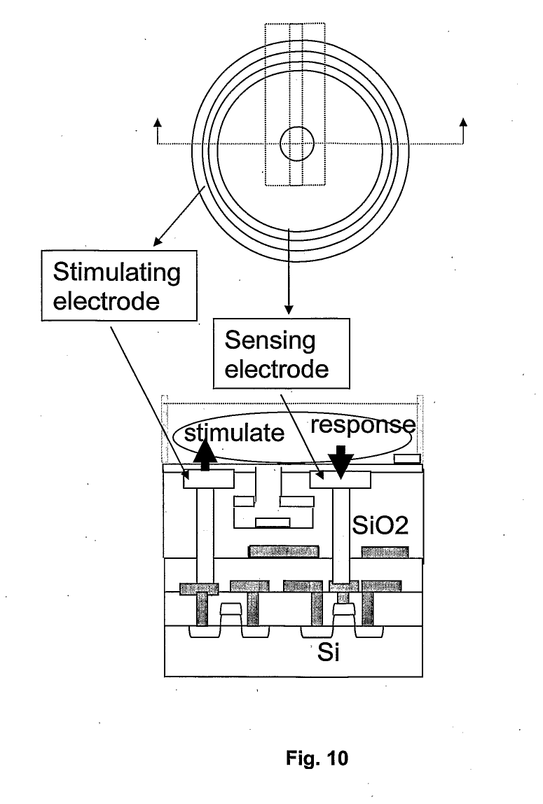

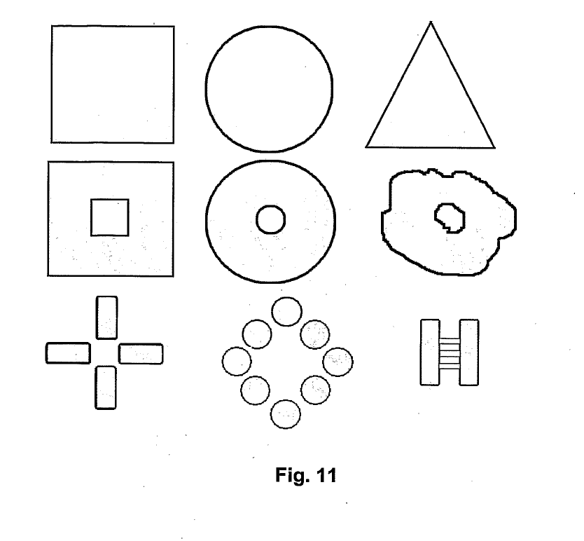

FIGS. 9 and 10 show two different embodiments of the biosensor of the invention, in which both stimulating electrodes and sensing electrodes are arranged within the sensing chamber. The sensing electrodes comprise a pair of electrically conductive plates, and the stimulating electrode comprise a similarly shaped pair of electrically conductive plates, said electrodes being arranged around the inlet 122 to the channel 121. In this embodiment, both pairs of electrodes are each shaped as a trapezoid. Alternatively, the sensing electrodes and the stimulating electrodes may be ring electrodes arranged in a mutually concentric manner, and with the inlet 122 present at around the centre of said rings. Some other alternative shapes of the sensing electrode and the stimulating electrode are shown in FIG. 11.

To summarize, the present invention and the embodiments thereof provides a biosensor, a method for forming a biosensor and a method of analysing the status of a biomolecule that has the following advantages:

1. automatically positioning of a biomolecule (e.g. a living cell like a neuron or a vesicle) on the biosensor;
2. easy stabilizing a single biomolecule or a plurality of bio-molecular bodies even on a biosensor having an array of regularly arranged electronic sensing elements during culturing and/or measuring bio-molecular bodies;
3. easy selecting bio-molecular bodies for pharmacological active ingredient screening; and
4. improving electrical coupling of bio-molecular bodies like neurons to the electronic sensing elements, e.g. the gate electrodes of transistors, by reducing the distance between each biomolecule and the respective electronic sensing element.

Although this invention has been described in terms of preferred embodiments, it has to be understood that numerous variations and modifications may be made, without departing from the spirit and scope of this invention as set out in the following claims.

1. A biosensor comprising:
a substrate having buried therein an electronic sensing element for sensing electrical variations due to a change in the status of a biomolecule, said substrate having a substrate surface located above the buried electronic sensing element;
a structured top layer covering the substrate surface, said structured top layer having a top surface located above the substrate surface, said top surface providing a sensing region on which a sample solution containing the biomolecule is placed,
a sensing electrode electrically coupled to the electronic sensing element, said sensing electrode having at least a part of its surface exposed to the sensing region, and
at least one channel for immobilizing the biomolecule via by suction through said channel, said channel being arranged in the structured top layer.

2. The biosensor of claim 1, wherein said channel comprises two openings terminating at the top surface of the structured top layer, one of said two openings being arranged proximate to the sensing electrode.

3. The biosensor of claim 1, wherein the top surface of the structured top layer is at least partially covered with a bio-compatible layer.

4. The biosensor of claim 1, wherein a patch clamp electrode is provided in the channel, said patch clamp electrode comprising a first and a second end, said first end of the patch clamp electrode being arranged proximate to one of said two openings, and said second end is connected to an amplifying circuit.

5. The biosensor of claim 1, wherein a reference electrode is arranged on the top surface of the structured top layer.

6. The biosensor of claim 1, wherein the sensing electrode is arranged between the top surface of the structured top layer and the substrate surface.

7. The biosensor of claim 1, wherein the sensing electrode is arranged on the top surface of the structured top layer.

8. The biosensor of claim 1, wherein the sensing electrode has a solid elliptical shape or a solid rectangular shape.

9. The biosensor of claim 1, wherein the sensing electrode has a ring (annular) an annular shape.

10. The biosensor of claim 9, wherein the first opening of the channel is arranged within the annular sensing electrode.

11. The biosensor of claim 1, wherein the top surface of the structured top layer is provided with chamber walls.

12. The biosensor of claim 1, wherein the chamber walls are arranged to define at least one sensing chamber.

13. The biosensor of claim 12, further comprising a fluid control chamber separated from the at least one sensing chamber by a chamber wall.

14. The biosensor of claim 13, wherein the first opening of the channel is arranged inside the at least one sensing chamber, and wherein a second opening of the channel is arranged inside the at least one fluid control chamber.

15. The biosensor of claim 1, further comprising a sensing electrode connector that is in physical contact with the sensing electrode and the electronic sensing element, thereby electrically coupling the sensing electrode to the electronic sensing element.

16. The biosensor of claim 1, wherein the substrate comprises a successively arranged layer sequence comprising at least a semiconductor layer, a first electrically insulating layer, a second electrically insulating layer and a third electrically insulating layer.

17. The biosensor of claim 16, wherein the electronic sensing element is arranged overlapping the semiconductor layer and the first electrically insulating layer.

18. The biosensor of claim 17, wherein the electronic sensing element is electrically connected to track conductors arranged in the second electrically insulating layer.

19. The biosensor of claim 18, wherein the sensing electrode is electrically coupled to the electronic sensing element via a sensing electrode connector arranged in the third electrically insulating layer.

20. The biosensor of claim 19, wherein the sensing electrode connection is surrounded by an electrically insulated screening electrode connected to ground and arranged in the third electrically insulating layer.

21. The biosensor of claim 16, wherein the electronic sensing element is a field effect transistor having a gate region, and wherein the sensing electrode is electrically connected to the gate region of the field effect transistor.

22. The biosensor of claim 21, wherein the sensing electrode is electrically connected to the gate region of the field effect transistor via the sensing electrode connector.

23. The biosensor of claim 22, wherein the substrate comprises an electrically insulated screening electrode connected to ground, said screening electrode being arranged around the sensing electrode connector.

24. The biosensor of claim 23, wherein the screening electrode is arranged in the third electrically insulating layer.

25. A method of forming a biosensor comprising:
providing a substrate having buried therein an electronic sensing element for sensing electrical variations due to a change in the status of a biomolecule, and a substrate surface located above the buried electronic sensing element;
covering the substrate surface with a structured top layer having a top surface above the substrate surface;
forming in or on the structured top layer a sensing electrode, the sensing electrode configured to sense electrical variations in the biomolecule;
forming in or on the structured top layer at least one channel for immobilizing the biomolecule via suction:
electrically coupling the sensing electrode to the electronic sensing element,
adapting the top surface for placing a biomolecule present in a sample solution thereupon; and
forming within the structured top layer at least one channel that is adapted to exert a suction force on the biomolecule.

26. The method of claim 25, wherein said channel comprises at least two openings terminating at the top surface, the channel being substantially buried in the structured top layer, one of the two openings being arranged proximate to the sensing electrode.

27. The method of claim 25, further comprising covering the top surface of the structure top layer with a bio-compatible layer.

28. The method of claim 25, further comprising providing a field effect transistor having a gate region as the electronic sensing element, and electrically connecting the sensing electrode to the gate region of the field effect transistor.

29. The method of claim 25, further comprising forming a sensing electrode connector in the substrate to electrically connect the sensing electrode to the gate region of the field effect transistor.

30. The method of claim 25, further comprising forming an electrically insulated screening electrode in the third electrically insulating layer of the substrate.

31. The method of claim 25, further comprising forming a patch clamp electrode in the channel.

32. The method of claim 25, further comprising forming a reference electrode on the top surface.

33. The method of claim 25, wherein forming a sensing electrode comprises the step of forming a solid elliptical or solid rectangular shaped sensing electrode.

34. The method of claim 25, wherein forming a sensing electrode comprises the step of forming a ring-shaped sensing electrode.

35. The method of claim 34, wherein forming a channel in the structured top layer comprises forming the first opening within the ring-shaped sensing electrode.

36. The method of claim 25, claim 25, further comprising covering the top surface of the structured top layer with chamber walls.

37. The method of claim 36, wherein covering the top surface with chamber walls comprises the steps of:
forming at least one sensing chamber, and
forming at least one sensing electrode in the at least one sensing chamber.

38. The method of claim 37, further comprising forming at least one fluid control chamber that is separated from the at least one sensing chamber via a chamber wall.

39. The method of claim 37, wherein forming at least one sensing chamber comprises the steps of:
forming the first opening of the channel inside the at least one sensing chamber, and
forming a second opening of the channel inside the fluid control chamber.

40. The method of claim 25, further comprising successively forming a layer sequence comprising a semiconductor layer, a first electrically insulating layer, a second electrically insulating layer and a third electrically insulating layer as the substrate.

41. The method of claim 40, further comprising forming the electronic sensing element overlapping the semiconductor layer and the first electrically insulating layer.

42. The method of claim 41, further comprising electrically connecting the electronic sensing element to track conductors arranged in the second electrically insulating layer.

43. The method of claim 42, further comprising the step of electrically coupling the sensing electrode to the electronic sensing element via a sensing electrode connection arranged in the third electrically insulating layer.

44. The method of claim 43, further comprising surrounding the sensing electrode connection by an electrically insulated screening electrode connected to ground and arranged in the third electrically insulating layer.

45. A method of analyzing the status of a biomolecule, comprising:
contacting the biomolecule with the sensing electrode of a biosensor as defined in claim 1,
measuring a first electrical signal associated with a first status of the biomolecule,
exposing the biomolecule to a condition that is suspected to be capable of changing the status of the biomolecule, and
measuring a second electrical signal that is associated with the status of the biomolecule after exposure to said condition.

46. The method of claim 45, further comprising
comparing the first and the second electrical signal to a pre-determined threshold electrical signal value for detecting the occurrence of a change in the status of the biomolecule.

47. The method of claim 46, wherein the shape and/or magnitude of the difference between the first and the second electrical signal is compared to the pre-determined threshold electrical signal value.

48. The method of claim 47, wherein when the shape and/or magnitude of the difference between the first and the second electrical signal is larger than those of the pre-determined threshold electrical signal value, the condition to which the biomolecule is exposed is evaluated to be capable of changing the status of the biomolecule.

49. The method of claim 45, further comprising immobilizing the biomolecule onto the biosensor by means of suction generated through the channel of the biosensor.

50. The method of claim 45, wherein said biomolecule comprises a cell which is able to undergo a change in its electrical potential.

51. The method of claim 50, wherein said cell comprises a eukaryotic cell.

52. The method of claim 51, wherein the eukaryotic cell is selected from a neuronal cell, an oocyte, a lymphocyte, a monocyte, a muscle cell, an embryonic stem cell and a yeast cell.

53. The method of claim 50, wherein said biomolecule comprises a prokaryotic cell.

54. The method of claim 53, wherein the prokaryotic cell is selected from the group consisting of archaea cells and bacteria cells.

55. The method of claim 50, claim 50, wherein measuring the first and/or second electrical signal comprises measuring an electrical current passing through a transport structure located within or isolated from the region of the cell on which the suction force is applied.

56. The method of claim 55, wherein said transport structure comprises anion channels, cation channels, anion transporters, cation transporters, receptor proteins and binding proteins.

57. The method of claim 45, wherein measuring the first electrical signal and/or second electrical signal comprises detecting an electrical characteristic of the biomolecule through the sensing element.

58. The method of claim 45, wherein measuring the first electrical signal and/or second electrical signal comprises measuring the electrical potential across the biomolecule, said electrical potential being measured between
a reference electrode present at the top surface of the biosensor and which is in contact with the sample solution, and
a patch clamp electrode located in a channel present in the structured top layer of the biosensor.

59. The method of claim 58, further comprising rupturing the surface of the biomolecule by means of the suction force, thereby allowing the electrical properties of transmembrane ion channels or of ionotropic receptors to be accessed.
